(19) United States
(12) Patent Application Publication
(10) Pub. No.: US 2015/0336917 A1
(43) Pub. Date: Nov. 26, 2015

(54) NOVEL PIPERAZINE COMPOUNDS, PHARMACEUTICAL COMPOSITIONS CONTAINING THE SAME AND METHODS OF TERMINATING ACUTE EPISODES OF CARDIAC ARRHYTHMIA, RESTORING NORMAL SINUS RHYTHM, PREVENTING RECURRENCE OF CARDIAC ARRHYTHMIA AND MAINTAINING NORMAL SINUS RHYTHM IN MAMMALS

(71) Applicant: CHANRX CORPORATION, Cleveland, OH (US)

(72) Inventor: Arthur M. BROWN, Brecksville, OH (US)

(73) Assignee: Laguna Pharmaceuticals, Inc., La Jolla, CA (US)

(21) Appl. No.: 14/437,485

(22) PCT Filed: Oct. 11, 2013
(86) PCT No.: PCT/US13/64640

§ 371 Date: Apr. 21, 2015

Related U.S. Application Data
(60) Provisional application No. 61/712,620, filed on Oct. 11, 2012.

(51) Int. Cl. C07D 295/10

(52) U.S. Cl. CPC C07D 295/10

(57) ABSTRACT

Novel piperazine compounds are disclosed. Also disclosed are pharmaceutical compositions containing these compounds and therapeutic methods of treating cardiac arrhythmias in mammals, particularly humans, including terminating acute episodes of cardiac arrhythmia, restoring normal sinus rhythm, preventing recurrence of cardiac arrhythmia and maintaining normal sinus rhythm using these compounds.

### BACKGROUND OF THE INVENTION

1. Field of the Invention

The present invention relates to novel piperazine compounds, pharmaceutical compositions containing the same and therapeutic methods of treating cardiac arrhythmias in mammals, particularly humans, including terminating acute episodes of cardiac arrhythmia, restoring normal sinus rhythm, preventing recurrence of cardiac arrhythmia and maintaining normal sinus rhythm.

2. Background of the Related Art

Human Ether-a-go-go Related Gene (hERG) is the pore-forming potassium channel subunit that underlies the cardiac repolarizing current I_{Kr} and consists of six transmembrane segments (S1-S6) and cytoplasmic amino- and carboxyl-termini. HERG has been linked to both congenital and drug-induced long QT syndrome, a serious and potential fatal heart condition.

Mutations in hERG produce functionally impaired channels and/or trafficking defective channels, both of which reduce I_{Kr} currents. Mutations spanning most of the molecule have been identified in different long QT families. This suggests that hERG plays a critical role in cardiac physiology.

Most of the drugs associated with long QT syndrome (drug-induced) are hERG blockers. See, e.g., Vandenberg et al., Trends Pharmacol. Sci. 22:240-246 (2001). Since the cardiotoxicity of the non-sedating antihistamine terfenadine (Seldane) was linked to hERG block in 1996 (see Roy et al., Circulation 94:817-823 (1996)), a wide variety of drugs having diverse structures, including antiarrhythmics, antibiotics, antipsychotics as well as antihistamines, have been shown to be potent hERG blockers.

Accordingly, hERG has become an important target for cardiac safety testing of new therapeutic agents. The US Food & Drug Administration currently recommends that pharmaceutical companies seeking approval for novel therapeutic compounds have them screened for potential hERG blocking.

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, they have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A third class of anti-arrhythmic action: effects on atrial and ventricular intracellular potentials and other pharmacological actions on cardiac muscle of MJ 1999 and AH 3747,” Br. J. Pharmacol 39:675-689 (1970), and Singh B. N., Vaughan Williams E. M., “The effect of amiodarone, a new anti-anginal drug, on cardiac muscle,” Br. J. Pharmacol 39:657-667 (1970)), but these are not selective Class III agents.

Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,”J. Am. Coll. Cardiol. 20:1063-1065 (1992)) Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two components of cardiac delayed rectifier K+ current. Differential sensitivity to block by Class III anti-arrhythmic agents,”J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A molecular basis for cardiac arrhythmia: hERG mutations cause long QT syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2,4′-piperidin]-6yl], (+)-, monochloride (MK-499) predominantly, if not exclusively, block I_{Kr}. Although, amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, is as a nonspecific adrenergic blocker, acts as an inhibitor of the enzyme phospholipase, and causes pulmonary fibrosis (Nademanee, K. “The Amiodarone Odessey”. J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness by prolonging APD, prevents and/or terminates reentrant arrhythmias. Most selective, Class III antiarrhythmic agents currently in development, such as d-sotalol and dofetilide predominantly, if not exclusively, block I_{Kr}, the rapidly activating component of I_{K} found both in atrium and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardigraphic QT interval, hence termed “acquired long QT syndrome,” has been observed when these compounds are utilized (Roden, D. M. “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supra-ventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced effectiveness and dangers of reverse use dependence,” Circulation, 81:686-690 (1990); Sadanaga et al., “Clinical evaluation of the use-dependent QRS prolongation and the reverse use-dependent QT prolongation of class III anti-arrhythmic agents and their value in predicting efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-dependent prolongation of cardiac action potentials by a methanesulfonanilide class III anti-arrhythmic agent: Specific block of rapidly activating delayed rectifier K+ current by dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

In view of the problems associated with current anti-arrhythmic agents, there remains a need for an effective treatment of cardiac arrhythmias in mammals.

### SUMMARY OF THE INVENTION

Accordingly, it is an object of the present invention to provide compounds and pharmaceutical compositions for preventing or treating cardiac arrhythmia in mammals, particularly humans.

In accordance with these and other objects, a first embodiment of the present invention comprises novel piperazine compounds having the structure:

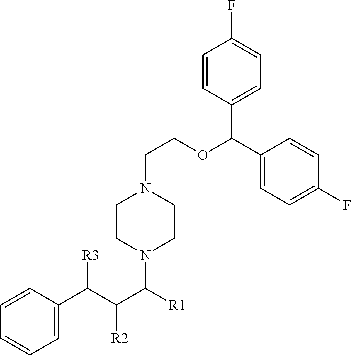

where each of R1, R2 and R3 is independently a hydrogen atom or a hydroxyl group, provided that not all of R1, R2 and R3 are the same and further provided that R1 and R2 are not both a hydroxyl group and R2 and R3 and not both a hydroxyl group.

A second embodiment of the present invention comprises pharmaceutical compositions containing one or more of the novel piperazine compounds shown above in admixture with a pharmaceutically acceptable carrier.

A third embodiment of the present invention comprises methods for terminating acute episodes of cardiac arrhythmia, such as atrial fibrillation or ventrical fibrillation, in a mammal, such as a human, by administering to that mammal at least one of the novel piperazine compounds shown above in an amount effective to terminate an acute episode of cardiac arrhythmia.

A fourth embodiment of the present invention is directed to a method for restoring normal sinus rhythm in a mammal, such as a human, exhibiting cardiac arrhythmia by administering at least one of the novel piperazine compounds shown above in an amount effective to restore normal sinus rhythm.

A fifth embodiment of the present invention is directed to a method for maintaining normal sinus rhythm in a mammal, such as a human, by administering at least one of the novel piperazine compounds shown above in an amount effective to maintain normal sinus rhythm in a mammal that has experienced at least one episode of cardiac arrhythmia.

A sixth embodiment of the present invention is directed to a method for preventing a recurrence of an episode of cardiac arrhythmia in a mammal, such as a human, by administering to that mammal at least one of the novel piperazine compounds shown above in an amount effective to prevent a recurrence of cardiac arrhythmia.

Additional advantages, objects and feature of the invention will be set forth in part in the description which follows and in part will become apparent to those having ordinary skill in the art upon examination of the following or may be learned from practice of the invention. The objects and advantages of the invention may be realized and attained as particularly pointed out in the appended claims.

### DETAILED DESCRIPTION OF PREFERRED EMBODIMENTS

A first preferred embodiment of the present invention comprises novel piperazine compounds having the structure:

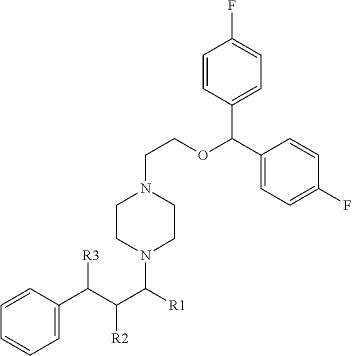

where each of R1, R2 and R3 is independently a hydrogen atom or a hydroxyl group, provided that not all of R1, R2 and R3 are the same and further provided that R1 and R2 are not both a hydroxyl group and R2 and R3 and not both a hydroxyl group. Particularly preferred compounds include those where only one of R1, R2 and R3 is a hydroxyl group. According to a particularly preferred embodiment, R1 is a hydroxyl group and R2 and R3 are each a hydrogen atom.

Pharmaceutically acceptable salts of the novel piperazine compounds may also be employed in the methods of the present invention. These pharmaceutically acceptable salts of include, but are not limited to, salts of vanoxerine formed from non-toxic inorganic or organic acids. Such pharmaceutically acceptable salts include, but are not limited to, the following: salts derived from inorganic acids, such as hydrochloric, hydrobromic, sulfuric, sulfamic, phosphoric, nitric and the like; salts derived from organic acids, such as acetic, propionic, succinic, glycolic, stearic, lactic, malic, tartaric, citric, ascorbic, pamoic, maleic, hydroxymaleic, phenylacetic, benzoic, salicylic, sulfanilic, 2-acetoxy-benzoic, fumaric, toluenesulfonic, methanesulfonic, ethane disulfonic, oxalic, isethionic, trifluoroacetic and the like; and salts derived from amino acids, such as glutamic add or aspartic acid. See U.S. Pat. No. 6,187,802 and WO 91/01732.

The novel piperazine compounds of the present invention and the pharmaceutically acceptable salts thereof can be synthesized by conventional chemical methods using starting materials and reagents known and available to those skilled in the art. For example, with respect to pharmaceutically acceptable slats, generally such salts are prepared either by ion exchange chromatography or by reacting the free base with stoichiometric amounts or with an excess of the desired salt-forming inorganic or organic acid in a suitable solvent or various combinations of solvents.

A second preferred embodiment of the present invention comprises pharmaceutical compositions containing one or more of the novel piperazine compounds shown above in admixture with a pharmaceutically acceptable carrier.

Such a pharmaceutical composition may be administered by any technique capable of introducing a pharmaceutically active agent to the desired site of action, including, but not limited to, buccal, sublingual, nasal, oral, topical, rectal and parenteral administration. Delivery of the compound may also be through the use of controlled release formulations in subcutaneous implants or transdermal patches.

For oral administration, a suitable composition containing a novel piperazine compound of the present invention, or a pharmaceutically acceptable salt thereof, may be prepared in the form of tablets, dragees, capsules, syrups and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of a novel piperazine compound of the present invention, or a pharmaceutically acceptable salt thereof. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing the a novel piperazine compound of the present invention, or a pharmaceutically acceptable salt thereof, and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, a mixture of a novel piperazine compound of the present invention, or a pharmaceutically acceptable salt thereof, and the desired additives may be filled into a capsule, such as a hard or soft gelatin capsule. The contents of a capsule and/or caplet may also be formulated using known methods to give sustained release of the active compound.

Liquid oral dosage forms of a novel piperazine compound of the present invention, or a pharmaceutically acceptable salt thereof, may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing a novel piperazine compound of the present invention, or a pharmaceutically acceptable salt thereof, may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of a novel piperazine compound of the present invention, or a pharmaceutically acceptable salt thereof, may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

A composition containing a novel piperazine compound of the present invention, or a pharmaceutically acceptable salt thereof, may also be administered nasally, for example by sprays, aerosols, nebulised solutions and/or powders. Metered dose systems known to those in the art may also be used.

Pharmaceutical compositions of a novel piperazine compound of the present invention, or a pharmaceutically acceptable salt thereof, may be administered to the buccal cavity (for example, sublingually) in known pharmaceutical forms for such administration, such as slow dissolving tablets, chewing gums, troches, lozenges, pastilles, gels, pastes, mouthwashes, rinses and/or powders.

Compositions containing a novel piperazine compound of the present invention, or a pharmaceutically acceptable salt thereof, for topical administration may comprise a matrix in which the pharmacologically active compound is dispersed such that it is held in contact with the skin in order to administer the compound transdermally. A suitable transdermal composition may be prepared by mixing a novel piperazine compounds of the present invention, or a pharmaceutically acceptable salt thereof, with a topical vehicle, such as a mineral oil, petrolatum and/or a wax, for example paraffin wax or beeswax, together with a potential transdermal accelerant such as dimethyl sulphoxide or propylene glycol.

Alternatively, a novel piperazine compound of the present invention, or a pharmaceutically acceptable salt thereof, may be dispersed in a pharmaceutically acceptable cream or ointment base. The amount of a novel piperazine compounds of the present invention, or a pharmaceutically acceptable salt thereof, contained in a topical formulation should be such that a therapeutically effective amount delivered during the period of time for which the topical formulation is intended to be on the skin.

A novel piperazine compound of the present invention, or a pharmaceutically acceptable salt thereof, may also be administered by continuous infusion either from an external source, for example by intravenous infusion or from a source of the compound placed within the body. Internal sources include implanted reservoirs containing the a novel piperazine compounds of the present invention, or a pharmaceutically acceptable salt thereof, to be infused which is continuously released for example by osmosis and implants which may be (a) liquid such as a suspension or solution in a pharmaceutically acceptable oil of the compound to be infused for example in the form of a very sparingly water-soluble derivative such as a dodecanoate salt or (b) solid in the form of an implanted support, for example of a synthetic resin or waxy material, for the compound to be infused. The support may be a single body containing all the compound or a series of several bodies each containing part of the compound to be delivered. The amount a novel piperazine compound of the present invention, or a pharmaceutically acceptable salt thereof, present in an internal source should be such that a therapeutically effective amount is delivered over a long period of time.

In addition, an injectable solution of a novel piperazine compound of the present invention, or a pharmaceutically acceptable salt thereof, can contain various additives such as preservatives, such as benzyl alcohol, methyl or propyl 4-hydroxybenzoate, benzalkonium chloride, phenylmercury borate and the like; as well as antioxidants, such as ascorbic acid, tocopherol, sodium pyrosulfate and optionally complex forming agents, such as an ethylenediamine tetraacetate salt for binding the metal traces, as well as buffers for adjusting the pH value and optionally a local anaesthetizing agent, e.g. lidocaine. The injectable solution containing a novel piperazine compound of the present invention, or a pharmaceutically acceptable salt thereof, is filtered before filling into the ampule and sterilized after filling.

A third preferred embodiment of the present invention comprises methods for terminating acute episodes of cardiac arrhythmia, such as atrial fibrillation or ventrical fibrillation, in a mammal, such as a human, by administering to that mammal at least one of the novel piperazine compounds in an amount effective to terminate an acute episode of cardiac arrhythmia.

A fourth preferred embodiment of the present invention comprises methods for restoring normal sinus rhythm in a mammal, such as a human, exhibiting cardiac arrhythmia by administering at least one of the novel piperazine compounds in an amount effective to restore normal sinus rhythm.

A fifth preferred embodiment of the present invention comprises methods for maintaining normal sinus rhythm in a mammal, such as a human, by administering at least one of the novel piperazine compounds in an amount effective to maintain normal sinus rhythm in a mammal that has experienced at least one episode of cardiac arrhythmia.

A sixth preferred embodiment of the present invention comprises methods for preventing a recurrence of an episode of cardiac arrhythmia in a mammal, such as a human, by administering to that mammal at least one of the novel piperazine compounds in an amount effective to prevent a recurrence of cardiac arrhythmia.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

What is claimed is:

1. A compound having the structure: 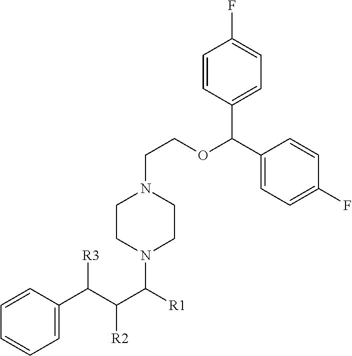
wherein each of R1, R2 and R3 is independently a hydrogen atom or a hydroxyl group, provided that not all of R1, R2 and R3 are the same and further provided that R1 and R2 are not both a hydroxyl group and R2 and R3 and not both a hydroxyl group.

2. The compound of claim 1, wherein only one of R1, R2 and R3 is a hydroxyl group.

3. The compound of claim 2, wherein R1 is a hydroxyl group and R2 and R3 are each a hydrogen atom.

4. A pharmaceutical composition comprising a compound of claim 1 in admixture with a pharmaceutically acceptable carrier.

5. A method for terminating acute episodes of cardiac arrhythmia in a mammal by administering to that mammal an effective amount of a compound of claim 1.

6. The method according to claim 5, wherein said cardiac arrhythmia is atrial fibrillation.

7. The method according to claim 5, wherein said cardiac arrhythmia is ventrical fibrillation.

8. The method according to claim 5, wherein said mammal is a human.

9. A method for preventing recurrence of an episode of cardiac arrhythmia in a mammal by administering to that mammal an effective amount of a compound of claim 1.

10. The method according to claim 9, wherein said mammal is a human.
